(19) United States
(12) Patent Application Publication
(10) Pub. No.: US 2025/0099761 A1
(43) Pub. Date: Mar. 27, 2025

(54) Stimulation of a Retina

(71) Applicant: Universität Duisburg-Essen, Körperschaft des öffentlichen Rechts, Essen (DE)

(72) Inventors: Philipp Löhler, Mülheim An Der Ruhr (DE); Andreas Erbslöh, Krefeld (DE)

(21) Appl. No.: 18/891,010

(22) Filed: Sep. 20, 2024

(30) Foreign Application Priority Data
Sep. 20, 2023 (DE) 10 2023 125 504.5

(51) Int. Cl. A61N 1/36; A61N 1/05

(52) U.S. Cl. CPC A61N 1/36046; A61N 1/0543; A61N 1/36139; A61N 1/36171

(57) ABSTRACT

Arrangement for stimulating a retina, comprising:
a camera,
electrodes for interacting with the retina,
an electronics configured
   to determine a respective target value for the stimulation of the retina for a multiplicity of pixels,
   to determine a respective set of stimulation parameters for each of the pixels,
   to output a stimulation signal to one of the electrodes for each of the pixels, in order to stimulate the retina,
   to receive action potentials, created by the retina, for each of the pixels,
   to determine an actual value of the stimulation of the retina for each of the pixels,
   to adjust the determination of the set of stimulation parameters for each of the pixels, in such a way that the actual value approaches the target value,
      wherein the electrodes are formed as part of a retinal implant of the arrangement.

### CROSS REFERENCE TO RELATED APPLICATIONS

This application claims priority to German Application No. 10 2023 125 504.5, filed on Sep. 20, 2023, the contents of which are hereby incorporated by reference in their entirety.

### FIELD OF INVENTION

The invention relates to an arrangement and a method for stimulating a retina.

### BACKGROUND

Retinal implants designed to provide persons gone blind with hazy vision are known. As a rule, such retinal implants have a multiplicity of electrodes for stimulating the retina. Electric signals are output to the retina via these electrodes. These signals are created via a chain of algorithms proceeding from a signal of a camera used to optically capture the surroundings.

Known retinal implants do not provide a satisfactory visual impression. In particular, the visual impression attainable with known retinal implants is often described as unnatural.

### SUMMARY

The problem addressed by the present invention is that of using retinal stimulation to obtain an improved visual impression in comparison with known retinal implants.

This problem is solved by the arrangement and the method according to the independent claims. Further advantageous configurations are indicated in the dependent claims. The features represented in the claims and in the description can be combined with one another in any technologically meaningful way.

According to the invention, an arrangement for stimulating a retina is presented. The arrangement comprises:
a camera,
a multiplicity of electrodes for interacting with the retina,
an electronics configured
   to use an image evaluation algorithm to determine a respective target value for the stimulation of the retina for a multiplicity of pixels from a camera signal output by the camera,
   to use a decoding algorithm to determine a respective set of stimulation parameters for each of the pixels on the basis of the determined target value,
   to output a stimulation signal to one of the electrodes for each of the pixels on the basis of the set of stimulation parameters, in order to stimulate the retina,
   to receive action potentials, created by the retina, for each of the pixels via one of the electrodes,
   to use a classification algorithm to determine an actual value of the stimulation of the retina for each of the pixels from the action potentials received,
   to use an optimization algorithm to adjust the determination of the set of stimulation parameters for each of the pixels on the basis of the determined target value and the determined actual value, in such a way that the actual value approaches the target value,
      wherein at least the multiplicity of electrodes are formed as part of a retinal implant of the arrangement.

A retina can be stimulated with electric signals using the described arrangement. Retinal ganglion cells, in particular, can be excited in the process. Other cell types can also be excited. A visual impression can be created by stimulating the retina. The described arrangement can be used for a person or for an animal. To this end, the arrangement is inserted, in full or in part, into an eye. Using the arrangement described, a fully or partially blind person or an animal can at least hazily perceive their surroundings optically. The arrangement interacts with the retina in the process. However, the retina is not part of the described arrangement.

The arrangement comprises a camera. The surroundings can be captured optically by means of the camera. This forms the basis of what is created as a visual impression. The camera can be a conventional camera. As customary for cameras, the camera outputs an electronic camera signal that represents information with regard to what has been optically captured by the camera.

Furthermore, the arrangement comprises a multiplicity of electrodes for interacting with the retina. Each electrode can be used to stimulate a corresponding part of the retina.

The electrodes are preferably arranged in an array. Hence, the retina can be stimulated in a two-dimensional grid such that a two-dimensional image is obtained as a visual impression. Like for a display, a resolution that is as high as possible is preferable here. However, a higher resolution requires a more complicated realization. For example, having a total number of electrodes in the range from 500 to 2000 has proven its worth. For example, the electrodes can be arranged in a 34×36 array. There are 1224 electrodes in that case.

Each electrode can correspond to a pixel of the image to be created as a visual impression. There is a one-to-one correspondence between pixel and electrode in that case. However, there is nothing preventing the combined use of a plurality of electrodes for a single pixel.

Furthermore, the arrangement comprises the electronics. This can be used to process the camera signal to form signals used to stimulate the retina via the electrodes. The electronics is preferably digital.

At least the multiplicity of electrodes is formed as part of a retinal implant. The retinal implant is part of the arrangement. Whether the electronics is also formed as part of the retinal implant in full or in part is irrelevant to the functionality of the arrangement described. Should the electronics not be formed as part of the retinal implant, the electronics can be formed outside of the retinal implant and connected to the retinal implant by way of a wireless connection or by way of a wired connection. Whether the camera is also formed as part of the retinal implant in full or in part is likewise irrelevant to the functionality of the arrangement described. Should the camera not be formed as part of the retinal implant, the camera can be formed outside of the retinal implant and connected to the retinal implant by way of a wireless connection or by way of a wired connection.

In particular, the electronics is configured to carry out the algorithms described in detail below. Whether these algorithms are executed on a single chip or on a plurality of chips connected to one another is irrelevant to the functionality of the arrangement described. In the simplest case, the electronics are formed as a chip that can be arranged within the person or animal.

The part of the electronics used to carry out the image evaluation algorithm can be referred to as image evaluator. The part of the electronics used to carry out the decoding algorithm can be referred to as decoder. The part of the electronics used to carry out the classification algorithm can be referred to as classifier. The part of the electronics used to carry out the optimization algorithm can be referred to as optimizer. However, whether the algorithms are executed on physically separate processors or executed as sub-algorithms using the same processor is irrelevant to the functionality of the arrangement.

The electronics is configured to use an image evaluation algorithm to determine a respective target value for the stimulation of the retina for the multiplicity of pixels from the camera signal output by the camera. The image evaluation algorithm is performed jointly for all pixels. The image evaluation algorithm obtains the camera signal as input. As output, the image evaluation algorithm outputs a respective target value for each of the pixels.

The target value is a measure for the extent to which the respective pixel should be excited. Numerous different definitions of the target value are conceivable. In the simplest case, the target value expresses how brightly the pixel should appear in the visual impression to be created. However, the nature of the retina is preferably considered within the definition of the target value. In particular, the target value can be a measure for the ratio with which the various retinal ganglion cells should be excited for the respective pixel. However, the target value can also simply be expressed as a percentage, wherein 0% can for example represent black and 100% can represent white. The image evaluation algorithm attempts to reproduce the functionality of the retinal image processing. For example, this can be carried out by implementing a difference of Gaussian (DoG) algorithm. In this case, a convolution is performed between a DoG kernel and the camera image, and this encodes either so-called ON or OFF cells by way of a sign change of the kernel. Changes over time that influence the activation rates can be identified by subtracting two obtained frames. The frames generated by DoG convolution and subtraction operator now lead to the desired activation ratios of the ON and OFF cells by virtue of the relationships between generated ON and OFF values being formed pixel-by-pixel.

However, the detailed form of the image evaluation algorithm is irrelevant to the functionality of the arrangement. It is sufficient that the image evaluation algorithm is suitable for determining a respective value for a multiplicity of pixels from the camera signal, said value being a measure for the sought after stimulation of the retina and, in this respect, being able to be considered a target value. In a simpler case, the image evaluation algorithm only establishes a respective brightness for each pixel and outputs this directly as target value.

The electronics is furthermore designed to use a decoding algorithm to determine a respective set of stimulation parameters for each of the pixels on the basis of the target value determined for this pixel by way of the image evaluation algorithm.

The decoding algorithm is carried out individually for each of the pixels. In the process, the decoding algorithm obtains the target value for this pixel as input. The set of stimulation parameters for this pixel is output by the decoding algorithm as output.

The decoding algorithm can be carried out successively for the individual pixels. Alternatively, the decoding algorithm can be carried out simultaneously for a portion of the pixels or else for all pixels.

The set of stimulation parameters contains the information as to how the retina should be excited for the corresponding pixel. The set of stimulation parameters is time-varying. Should the image of the surroundings captured by the camera change, the respective set of stimulation parameters generally also changes for each of the pixels. This allows a visual impression to be obtained in real-time.

For example, the decoding algorithm can be implemented as a neural network. The latter can calculate corresponding stimulation parameters from input parameters for so-called ON and OFF activation ratios. Different layer types can be used to this end, e.g. a dense (fully connected) layer, convolution layer or LSTM layer. For example, this decoder network can be trained by forming a Loss function which represents an error between ACTUAL data (classifier output) and TARGET data (image processing output) as “crossentropy loss”.

However, the detailed form of the decoding algorithm is irrelevant to the functionality of the arrangement. It is sufficient that the decoding algorithm is suitable for determining a respective set of parameters, which can be considered to be stimulation parameters, for each of the pixels on the basis of the target value determined for this pixel using the image evaluation algorithm. In particular, it is not mandatory for the decoding algorithm to be formed as a neural network. In a simpler case, the decoding algorithm merely converts, for each pixel, a respective brightness value into an amplitude for the stimulation signal.

The electronics is furthermore designed to output a stimulation signal to one of the electrodes for each of the pixels on the basis of the set of stimulation parameters determined for this pixel using the decoding algorithm, in order to stimulate the retina. This can be carried out successively for the individual pixels. Alternatively, this can be carried out simultaneously for a portion of the pixels or else for all pixels.

The stimulation signal is an electric signal. The stimulation signal acts on the retina and stimulates the latter. As a result, the cells of the retina are activated, and so a corresponding visual impression arises. The set of stimulation parameters characterizes the stimulation signal. A visual impression is composed from the individual pixels and in any case hazily corresponds to how the camera captures the surroundings.

The arrangement described here does not only allow stimulation of the retina as described. Additionally, the described arrangement can also capture how well this is carried out. The described arrangement thus enables feedback about the retinal stimulation. This can assess the stimulation success. The result obtained in the process can be considered when determining the stimulation parameters. As a result, the stimulation can also be referred to as an “adaptive closed-loop stimulation”.

Stimulation of the retina leads to the creation of action potentials by the retina. Ultimately these bring about the visual impression. The invention is based on the insight that these action potentials can be measured and used as feedback.

The electronics is configured to receive action potentials, created by the retina, for each of the pixels via one of the electrodes. This can be carried out successively for the individual pixels. Alternatively, this can be carried out simultaneously for a portion of the pixels or else for all pixels.

The stimulation signal for a pixel can be output via the same electrodes that are also used to capture the action potentials, created by the retina, for this pixel. In this case, the electrodes can also be referred to as bidirectional. However, the same functionality can also be obtained if the action potentials are captured by electrodes provided solely for this purpose. Against the background of these general considerations there are the three embodiments described below.

In a first preferred embodiment, the electronics is configured to output a stimulation signal to one of the electrodes for each of the pixels on the basis of the set of stimulation parameters, in order to stimulate the retina, and to receive action potentials, created by the retina, from this electrode. In this embodiment, the stimulation signal for a pixel is thus output to the electrode that is also used to receive the action potentials.

In a second preferred embodiment, the electronics is configured to output a stimulation signal to one of the electrodes for each of the pixels on the basis of the set of stimulation parameters, in order to stimulate the retina, and to receive action potentials, created by the retina, from another one of the electrodes. In this embodiment, the stimulation signal for a pixel is thus output to a first of the electrodes, while the action potentials for this pixel are received by a second electrode. The second electrode is preferably adjacent to the first electrode. There can be a plurality of second electrodes. For example, the stimulation signal for a pixel can be output to a first of the electrodes, while the action potentials for this pixel are received by a plurality of second electrodes that surround the first electrode.

In the second embodiment, the stimulation signals can be output temporally independently of the reception of the action potentials as a matter of principle. However, the individual electrodes can also fulfil different functions in succession. All electrodes are preferably used to emit stimulation signals in order to be able to create a visual impression with a resolution that is as high as possible. In the second embodiment, this can be realized, for example, by virtue of the individual electrodes each being used in alternation, first as a first electrode for one of the pixels and subsequently as a second electrode for a different one of the pixels.

In a third preferred embodiment, one portion of the pixels are treated in accordance with the first embodiment and another portion of the pixels are treated in accordance with the second embodiment.

The fact that the stimulation signal for a pixel is output at one of the electrodes generally means that the stimulation signal for a pixel is output at at least one of the electrodes. There can also be a plurality of electrodes. The fact that the action potentials for a pixel are received via one of the electrodes generally means that the action potentials for a pixel are received via at least one of the electrodes. There can also be a plurality of electrodes.

To the extent this affects the stimulation of the retina by the electrodes, the electrodes could be jointly considered as a stimulator. To the extent this affects the reception of the action potentials from the retina, the electrodes could be jointly considered as a recorder. However, since the electrodes are preferably used for both of these purposes in each case, such a summary label for the electrodes is dispensed with herein. The electrodes are preferably formed jointly on a chip which can be inserted into the eye.

In any case, respective action potentials are received for each of the pixels. These can be used as feedback and processed further accordingly. To this end, the electronics is configured to use a classification algorithm to determine a respective actual value of the stimulation of the retina for each of the pixels from the action potentials received for this pixel. The classification algorithm can be carried out successively for the individual pixels. Alternatively, the classification algorithm can be carried out simultaneously for a portion of the pixels or else for all pixels.

Preferably, the classification algorithm is formed as a neural network. The latter can consist of e.g. dense (fully connected) layers and be trained separately from the decoder algorithm. For training purposes, recordings of neuronal reactions to irradiation with pulsed light can be used. In the process, cells that emit action potentials more frequently as a result of the optical irradiation are labelled ON cells and cells which have more action potentials in darkness are labelled OFF cells. This labelling enables what is known as supervised learning, by means of which the weights of the neural network can be trained such that a correct assignment of the respective cell type by way of the network is made possible. The network pre-trained thus can now be used in the degenerated retina in order to correctly assign the action potentials, evoked by electric stimulation, to one of the cell types.

However, the detailed form of the classification algorithm is irrelevant to the functionality of the arrangement. It is sufficient that the classification algorithm is able to classify the received action potentials in such a way that it is possible to obtain a value which is a measure for the actual excitation of the retina. Accordingly, this value can be considered to be the actual value. In particular, it is not mandatory for the classification algorithm to be formed as a neural network. In a simple case, the classification algorithm might also merely output a value that is a measure for the strength of the action potentials received. A neural network is not required to this end.

The action potentials created by the retina are comparatively weak and generally of the order of μV. By preference, the electronics is configured to amplify the action potentials created by the retina. Amplification can be carried out as part of the classification algorithm or in advance of the classification algorithm.

In the ideal case, the actual value determined for a specific pixel using the classification algorithm corresponds to the target value determined for this pixel using the image evaluation algorithm; ideally, this applies permanently to all pixels. As conventional for a target/actual comparison, the actual value is defined in a manner analogous to the target value. Thus, the actual value and the target value each represent a value of the same parameter. This renders a comparison of these values possible.

Using the described arrangement it is possible not only to capture but also correct deviations from this ideal state. To this end, the electronics is configured to use an optimization algorithm to adjust the determination of the set of stimulation parameters for each of the pixels on the basis of the target value determined for this pixel using the image evaluation algorithm and the actual value determined for this pixel using the classification algorithm.

Thus, in the case of a deviation between actual value and target value, it is possible to intervene in the conversion of the target value into the set of stimulation parameters, on an individual basis for each of the pixels. Hence, an improved visual impression overall is obtained vis-à-vis known retinal implants without such feedback.

The determination of the set of stimulation parameters is adjusted in such a way that the actual value approaches the target value. Hence, the error between actual and target value is reduced. Thus, what is achieved as a result of the described feedback is that the retina is reliably excited as desired. This optimization is implemented continually and for each of the pixels. Overall, this provides a visual impression which could not be obtained without this feedback.

The target value determined for a pixel is a measure of how this pixel should be represented in the created visual impression. Should the action potentials detected for this pixel reveal that the retina has been excited more strongly for this pixel than desired, the optimization algorithm is used to intervene in the determination of the set of stimulation parameters such that this pixel is excited to a lesser extent. For example, this can be implemented by virtue of the decoding algorithm outputting a smaller amplitude of the stimulation signal for this pixel as part of the set of stimulation parameters. In turn, this can be implemented by adapting the weights used in the decoding algorithm. Thus, the decoding algorithm can be influenced in such a way for this pixel that the decoding algorithm tends to output a smaller amplitude for this pixel. A corresponding statement applies for the case in which the retina has been excited to a lesser extent for this pixel than desired.

The decoding algorithm is used to convert the desired excitation of the retina into stimulation parameters. How close the actual excitation of the retina comes to the desired excitation is a question of calibration as a matter of principle. As a result of the described feedback, this calibration can be corrected not only continuously. Moreover, this adaptation of the calibration is also implemented pixel-by-pixel. Hence, the excitation of the individual pixels relative to one another can be set particularly well. A visual impression with a particularly well set excitation of the pixels relative to one another is a good visual impression.

The optimization algorithm is used to intervene in the decoding algorithm. Preferably, the decoding algorithm is the only one of the described algorithms that can be changed.

Different algorithms can be used as optimization algorithm, e.g. the Adams optimizer. Should the decoding algorithm be in the form of a neural network, the optimization algorithm can be designed to train the neural network. In that case in particular, the optimization algorithm can also be referred to as an “optimizer” or as a “training optimizer”. These terms are conventional in the context of AI. What is common to most optimizers is that they minimize a predefined loss function (e.g. categorical crossentropy) by way of a gradient descent method. Should the decoder algorithm be formed as a neural network, this is implemented by adapting the neuron weights in the decoder algorithm until a minimum is found.

However, the detailed form of the optimization algorithm is irrelevant to the functionality of the arrangement. It is sufficient that the optimization algorithm is able to act on the decoding algorithm in such a way that the actual value approaches the target value. The decoding algorithm does not need to be a neural network, and the optimization algorithm does not need to represent a training optimizer for this neural network either.

In a preferred embodiment of the arrangement, the determined target value for the stimulation of the retina expresses the ratio with which the ON ganglion cells and OFF ganglion cells of the retina are to be excited.

Previous retinal implants had neither clinical nor commercial success since the evoked visual impressions only contained phosphenes and were qualitatively incomparable with natural vision. The reason therefor lies in the non-selective activation of the different ganglion cell types in the retina. Such a non-selective activation does not correspond to the natural processes in the retina. In the healthy eye, different subclasses of the ganglion cells encode different light conditions. The so-called OFF ganglion cells encode dark edge portions while conversely so-called ON ganglion cells encode bright edge portions. This is accounted for in the present embodiment. To this end, the image evaluation algorithm is used to determine, as a target value for each of the pixels, the ratio with which the ON ganglion cells and the OFF ganglion cells of the retina are to be excited for this pixel. An improved visual impression can be obtained as a result.

Within this embodiment, it is preferable that the actual value of the stimulation of the retina determined using the classification algorithm expresses the ratio with which ON ganglion cells and OFF ganglion cells of the retina were excited. As a result, actual value and target value are defined in a manner analogous to one another. The described definition of the actual value is possible because the neurons of the retina emit signals with a characteristic shape. As a result, it is possible to distinguish whether a specific signal emitted by the retina was created by an ON ganglion cell or by an OFF ganglion cell. It follows that the ratio of these signals can be determined.

In a further preferred embodiment of the arrangement, the decoding algorithm is in the form of a neural network, wherein the optimization algorithm is configured to influence weights of the decoding algorithm.

It transpired that the use of a neural network for the decoding algorithm is particularly advantageous because the retina is also a neural network. As a result of the similarity of the networks, the retina can be incorporated into the data processing particularly well in this embodiment. In this case, the optimization algorithm can intervene in the decoding algorithm by virtue of the optimization algorithm influencing the weights of the decoding algorithm such that the actual value approaches the target value. The weights of the decoding algorithm can also be referred to as neuron weights. Should the received action potentials yield that the retina is excited more strongly in a pixel than desired, the decoding algorithm can be influenced by way of the optimization algorithm, in such a way that the decoding algorithm outputs a set of stimulation parameters for this pixel, with the set corresponding to a weaker excitation of the retina. A corresponding statement applies if the retina is excited to a lesser extent in a pixel than desired.

In a further preferred embodiment of the arrangement, the stimulation signal is a superposition of a plurality of sinusoidal signals, which each have an individually selectable amplitude and/or an individually selectable frequency. This applies pixel-by-pixel. This means that a plurality of sinusoidal signals are in each case superimposed for each of the pixels in order to obtain the stimulation signal for this pixel.

In this embodiment, the set of stimulation parameters contains one or more stimulation parameters for each of the sinusoidal signals. For example, the set of stimulation parameters may in each case contain an amplitude, a frequency and a repetition rate for each of the sinusoidal signals. In this case, the individual amplitudes can be between 0 and 100%, in relation to a reference quantity. Consequently, the ratio with which the individual sinusoidal signals are superimposed can be defined by way of the amplitudes.

The visual impression becomes ever more realistic the more sinusoidal signals are superimposed on one another. However, the outlay also increases with the number of sinusoidal signals to be superimposed. In practice, it has proven its worth that the stimulation signal is a superposition of two to six sinusoidal signals, in particular of four sinusoidal signals.

The present embodiment is based on the insight that the retina reacts to a stimulation with a selective response. This applies in particular to a stimulation in the kHz range with an amplitude in the μA range. Accordingly, it is preferable, especially in this embodiment, for the frequencies to in each case lie between 1 and 10 kHz and/or for the amplitudes to be in the range of 0 to 200 μA. The selective response means that the functional cell classes which encode different impressions can be activated in isolation by an electrical stimulation. The visual impression obtained thereby comes close to the natural visual impression of a healthy eye.

Moreover, it was recognized that the waveform of the stimulation signal also plays a role in the activation quality. Conventional retinal implants are based on stimulation patterns with rectangular current pulses. By contrast, the sinusoidal stimulation waveforms used in the present embodiment lead to a substantially more robust cell activation.

Preferably, the stimulation signals for the individual electrodes are created using a signal generator which has a respective output for each of the electrodes. For example, the signal generator may comprise 1224 output channels in order to realize a resolution of 34×36. For each of the output channels, the signal generator can be used to create a respective mixture of e.g. four freely selectable sinusoidal frequencies in the range between 1 and 10 kHz. The respective amplitude of these four sinusoidal waves can likewise be defined freely for each frequency, with the result that very versatile stimulation waveforms are possible. An individual frequency mix can be output on each of the output channels, bringing about a high resolution of the evoked visual impression.

Present retinal stimulators are incapable of generating both the high frequency in the kHz range and the required sinusoidal waveform, and of using these simultaneously for the stimulation on independent output channels. Although stimulators that can generate arbitrary signal forms exist, they have only very few output channels, which cannot reproduce the high resolution of natural vision, or are only able to output the output signal in temporally asynchronous fashion by way of multiplexers. Additionally, these are not suitable for human implantation on account of the high data rate required.

In the arrangement described, use is preferably made of a retinal stimulator which has available a division into globally available and locally available circuit constituents. The waveforms with their different frequencies can be generated in the global circuit part by way of digital-to-analogue converters, while the amplitude modulation of the individual frequencies can be implemented locally by way of capacitively stored voltage values at the pixels. The capacitively stored voltages can control the reference current of operational transimpedance amplifiers (OTAs), enabling an amplitude modulation of the sinusoidal signals which are homogeneous in terms of their amplitude. This can reduce the required data rate and the circuit complexity without having to accept a deterioration in signal quality or synchronicity. The above-descried restrictions were overcome by this configuration.

In a further preferred embodiment of the arrangement, the set of stimulation parameters comprises an amplitude, a frequency and a repetition rate.

In this embodiment, a respective amplitude, frequency and repetition rate are determined for each of the pixels using the decoding algorithm. These parameters are summarily labelled as the set of stimulation parameters for this pixel.

In this embodiment, the stimulation signal is output to the retina in pulsed form. In this context, the repetition rate specifies how quickly the pulses are emitted to the retina in succession.

A further aspect of the invention presents a method for stimulating a retina with a multiplicity of electrodes for interacting with the retina, wherein at least the multiplicity of electrodes are formed as part of a retinal implant and wherein:
a) an image evaluation algorithm is used to determine a respective target value for the stimulation of the retina for a multiplicity of pixels from a camera signal output by a camera,
b) a decoding algorithm is used to determine a respective set of stimulation parameters for each of the pixels on the basis of the determined target value,
c) a stimulation signal is output to one of the electrodes for each of the pixels on the basis of the set of stimulation parameters, in order to stimulate the retina,
d) action potentials created by the retina are received for each of the pixels via one of the electrodes,
e) a classification algorithm is used to determine an actual value of the stimulation of the retina for each of the pixels from the action potentials received,
f) an optimization algorithm is used to adjust the determination of the set of stimulation parameters for each of the pixels on the basis of the determined target value and the determined actual value, in such a way that the actual value approaches the target value.

The advantages and features of the arrangement can be applied and transferred to the method, and vice versa. The arrangement is preferably configured for being operated according to the method. The method is preferably carried out with the arrangement.

The invention is explained in more detail below on the basis of the FIGURE. The FIGURE shows a particularly preferred exemplary embodiment, to which the invention is not restricted however. The FIGURE and the relative sizes shown therein are only schematic. In detail:

### BRIEF DESCRIPTION OF THE DRAWINGS

FIG. 1: shows an arrangement according to the invention for stimulating a retina.

### DETAILED DESCRIPTION

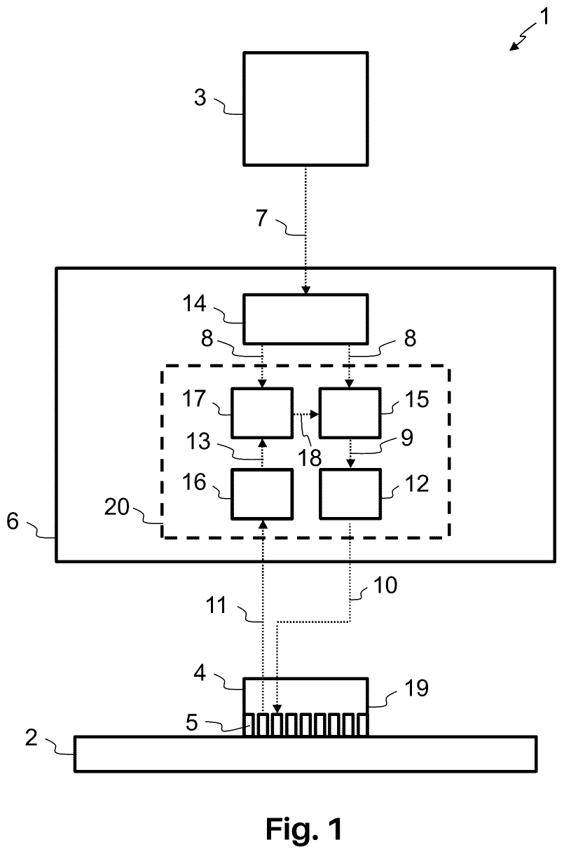

FIG. 1 shows an arrangement 1 for stimulating a retina 2. The retina 2 is also plotted in FIG. 1 but not part of the arrangement 1. The arrangement 1 comprises a camera 3. The surroundings are captured optically by means of the camera 3. The camera 3 outputs a camera signal 7 with appropriate information.

Furthermore, the arrangement 1 comprises an electronics 6 and a multiplicity of electrodes 5 for interacting with the retina 2. The electrodes 5 are part of a chip 4 which rests against the retina 2. The electronics 6 is used to process the camera signal 7 into signals that are output to the electrodes 5. As a result, the retina 2 is stimulated in such a way that this give rise to a visual impression reproducing the surroundings captured by the camera.

FIG. 1 plots nine electrodes 5 by way of example, next to one another for reasons of simplicity. Only one of the electrodes 5 has been provided with a reference sign. In reality, the electrodes 5 are preferably arranged in a two-dimensional array. This allows a two-dimensional image to be created.

The multiplicity of electrodes 5 are formed as part of a retinal implant 19. The retinal implant 19 is part of the arrangement 1. Whether the camera 3 and the electronics 6 is also formed as part of the retinal implant 19 in full or in part is irrelevant to the functionality of the arrangement 1 described.

The electronics 6 is configured to use an image evaluation algorithm 14 to determine a respective target value 8 for the stimulation of the retina 2 for a multiplicity of pixels from the camera signal 7 output by the camera 3. The determined target value 8 for the stimulation of the retina 2 expresses the ratio with which ON ganglion cells and OFF ganglion cells of the retina 2 should be excited for this pixel.

The image evaluation algorithm 14 is carried out jointly for all pixels. The algorithms described below are carried out using the electronics 6, in each case on an individual basis for each of the pixels. In FIG. 1, this is depicted in as much as, by way of example, an algorithm block 20 is shown for one of the pixels. This algorithm block 20 interacts with two of the electrodes 5. Further identical algorithm blocks 20 are provided for the remaining electrodes 5. However, only one of the algorithm blocks 20 has been shown for the sake of clarity.

The algorithm block 20 comprises a decoding algorithm 15. Hence, a respective set of stimulation parameters 9 can be determined for each of the pixels on the basis of the determined target value 8.

The electronics 6 is configured to output a stimulation signal 10 to one of the electrodes 5 for each of the pixels on the basis of the set of stimulation parameters 9, in order to stimulate the retina 2. To this end, in the shown example of FIG. 1, the set of stimulation parameters 9 is converted to form the stimulation signal 10 using a signal generator 12. The stimulation signal 10 is a superposition of a plurality of sinusoidal signals, which each have an individually selectable amplitude and an individually selectable frequency. The set of stimulation parameters 9 comprises an amplitude, a frequency and a repetition rate for each of these sinusoidal signals.

The electronics 6 is furthermore configured to receive action potentials 11, created by the retina 2, for each of the pixels via one of the electrodes 5. In the example of FIG. 1, this is implemented by means of an electrode 5 located next to the electrode 5 that receives the stimulation signal 10 for this pixel. Alternatively, however, the action potentials 11 could for example be received by the same electrode 5 that also receives the stimulation signal 10 for this pixel.

The algorithm block 20 furthermore comprises a classification algorithm 16. From this, an actual value 13 of the stimulation of the retina 2 can be determined from the action potentials 11 received.

The algorithm block 20 furthermore comprises an optimization algorithm 17. This can be used to adjust the determination of the set of stimulation parameters 9 on the basis of the determined target value 8 and the determined actual value 13, in such a way that the actual value 13 approaches the target value 8. To this end, an appropriate correction signal 18 can be output to the decoding algorithm 15 by the optimization algorithm 17. The correction signal 18 brings about a change of the weights in the decoding algorithm 15.

### LIST OF REFERENCE SIGNS

1 Arrangement
2 Retina
3 Camera
4 Chip
5 Electrodes
6 Electronics
7 Camera signal
8 Target value
9 Set of stimulation parameters
10 Stimulation signal
11 Action potentials
12 Signal generator
13 Actual value
14 Image evaluation algorithm
15 Decoding algorithm
16 Classification algorithm
17 Optimization algorithm
18 Correction signal
19 Retinal implant
20 Algorithm block

1. Arrangement for stimulating a retina, comprising:
a camera,
a multiplicity of electrodes for interacting with the retina,
an electronics configured
to use an image evaluation algorithm to determine a respective target value for the stimulation of the retina for a multiplicity of pixels from a camera signal output by the camera,
to use a decoding algorithm to determine a respective set of stimulation parameters for each of the pixels on the basis of the determined target value,
to output a stimulation signal to one of the electrodes for each of the pixels on the basis of the set of stimulation parameters, in order to stimulate the retina,
to receive action potentials, created by the retina, for each of the pixels via one of the electrodes,
to use a classification algorithm to determine an actual value of the stimulation of the retina for each of the pixels from the action potentials received,
to use an optimization algorithm to adjust the determination of the set of stimulation parameters for each of the pixels on the basis of the determined target value and the determined actual value, in such a way that the actual value approaches the target value,
wherein at least the multiplicity of electrodes are formed as part of a retinal implant of the arrangement.

2. Arrangement according to claim 1, wherein the determined target value for the stimulation of the retina expresses the ratio with which the ON ganglion cells and OFF ganglion cells are to be excited.

3. Arrangement according to claim 1, wherein the decoding algorithm is in the form of a neural network and wherein the optimization algorithm is configured to influence weights of the decoding algorithm.

4. Arrangement according to claim 1, wherein the stimulation signal is a superposition of a plurality of sinusoidal signals, which each have an individually selectable amplitude and/or an individually selectable frequency.

5. Arrangement according to claim 1, wherein the set of stimulation parameters comprises an amplitude, a frequency and a repetition rate.

6. Method for stimulating a retina with a multiplicity of electrodes for interacting with the retina, wherein at least the multiplicity of electrodes are formed as part of a retinal implant and wherein:
a) an image evaluation algorithm is used to determine a respective target value for the stimulation of the retina for a multiplicity of pixels from a camera signal output by a camera,
b) a decoding algorithm is used to determine a respective set of stimulation parameters for each of the pixels on the basis of the determined target value,
c) a stimulation signal is output to one of the electrodes for each of the pixels on the basis of the set of stimulation parameters, in order to stimulate the retina,
d) action potentials created by the retina are received for each of the pixels via one of the electrodes,
e) a classification algorithm is used to determine an actual value of the stimulation of the retina for each of the pixels from the action potentials received,
f) an optimization algorithm is used to adjust the determination of the set of stimulation parameters for each of the pixels on the basis of the determined target value and the determined actual value, in such a way that the actual value approaches the target value.

7. Arrangement according to claim 2, wherein the decoding algorithm is in the form of a neural network and wherein the optimization algorithm is configured to influence weights of the decoding algorithm.

8. Arrangement according to claim 2, wherein the stimulation signal is a superposition of a plurality of sinusoidal signals, which each have an individually selectable amplitude and/or an individually selectable frequency.

9. Arrangement according to claim 3, wherein the stimulation signal is a superposition of a plurality of sinusoidal signals, which each have an individually selectable amplitude and/or an individually selectable frequency.

10. Arrangement according to claim 2, wherein the set of stimulation parameters comprises an amplitude, a frequency and a repetition rate.

11. Arrangement according to claim 3, wherein the set of stimulation parameters comprises an amplitude, a frequency and a repetition rate.

12. Arrangement according to claim 4, wherein the set of stimulation parameters comprises an amplitude, a frequency and a repetition rate.
